(19) United States
(12) Patent Application Publication
(10) Pub. No.: US 2010/0003279 A1
(43) Pub. Date: Jan. 7, 2010

(54) VACCINE FOR IN OVO INOCULATION

(75) Inventors: Ohta Hideyuki, Kumamoto-ken (JP); Shinsuke Ezoe, Kumamoto-ken (JP); Kenichi Yamazaki, Kumamoto-ken (JP); Toru Kawai, Kumamoto-ken (JP)

Correspondence Address: BROWDY AND NEIMARK, P.L.L.C.;624 NINTH STREET, NW, SUITE 300, WASHINGTON, DC, 20001-5303, US

(73) Assignee: JURIDICAL FOUNDATION THE CHEMO-SERO-THERAPEUTIC RESEARCH INSTITUTE, Kumamoto-shi, Kumamoto-ken (JP)

(21) Appl. No.: 12/158,787

(22) PCT Filed: Dec. 21, 2006
(86) PCT No.: PCT/JP2006/325517

§ 371 Date: Jun. 23, 2008

(30) Foreign Application Priority Data
Dec. 22, 2005 (JP) 2005-370982

(51) Int. Cl. A61K 39/21; A61K 39/12; A61K 39/275; A61K 39/255; A61K 39/125; A61K 39/235; A61P 31/12; A61K 39/17; A61K 39/215; A61K 39/145; A61K 39/15

(52) U.S. Cl. 424/207.1; 424/204.1; 424/232.1; 424/229.1; 424/216.1; 424/233.1; 424/214.1; 424/222.1; 424/209.1; 424/215.1

(57) ABSTRACT

A purpose of the present invention is to provide a vaccine for in ovo inoculation effective for prevention of any fowl viral diseases. A fowl vaccine for in ovo inoculation with high efficacy in view of safety as well wherein, by holding such live viruses on a virus-adsorbing agent through adsorption that have been difficult for practical usage as a vaccine for in ovo inoculation, viral growth in embryonated chicken eggs after in ovo inoculation is retarded to thereby reduce pathogenicity of the viruses to embryo to avoid reduction in hatching rate and to alleviate severity in clinical symptoms after hatching. A virus-adsorbing agent to be used in the vaccine includes an aluminum compound such as aluminum hydroxide gel, potassium alum and the like.

### TECHNICAL FIELD

The present invention relates to a fowl vaccine for in ovo inoculation. More specifically, the present invention relates to a fowl vaccine for in ovo inoculation characterized by that live viruses are held on a virus-adsorbing agent through adsorption.

### BACKGROUND ART

Commercial fowls bred all over the world are injected with a vaccine for protection from infection or pathogeny with various pathogens. Viruses that commonly develop diseases in fowls include Marek's disease virus, fowl pox virus, infectious bursal disease virus, Newcastle disease virus, infectious bronchitis virus, infectious laryngotracheitis virus, avian encephalomyelitis virus, chicken anemia virus, avian influenza virus, avian reovirus, avian leukemia virus, reticuloendotheliosis virus, fowl adenovirus, avian pneumovirus, pigeon pox virus, and the like. Effects of these diseases on fowl industry are immense. Currently, a vaccine is utilized against these numerous diseases.

In general, a vaccine may be divided into a live vaccine and an inactivated vaccine. A live vaccine is one usually with a strain attenuated by artificial procedure or with a natural attenuated strain. On the other hand, an inactivated vaccine is a vaccine wherein infectivity of a virus is removed by a physical or chemical treatment. With a live vaccine, a local immunization may be expected at an earlier stage since it may be administered via natural infectious route, both humoral and cellular immunity may be established and the duration of immunity is in general prolonged. On the contrary, with an inactivated vaccine, humoral immunization may primarily be established but at a later stage and with a shorter duration of immunity and thus an adjuvant is added in most cases for the purpose of attaining a prolonged duration of immunity.

When a live vaccine is used, as affected with maternal antibodies, a plural vaccination is commonly done in consideration of dispersion of maternal antibodies. Also, a level of maternal antibodies may vary with to where a hatching egg is introduced and thus a vaccine program needs to be appropriately designed depending on each of the maternal antibodies. There are strains with a variety of pathogenicity in respective vaccines, which are selected based on the state of contamination in the field and the level of maternal antibodies. Under these circumstances, designing an optimal vaccine program would require considerable knowledge and experience and hence is not easy in general.

As described above, effects of the various diseases on fowl industry are immense. For instance, when fowls are infected with pathogenic strains in the field before acquiring suitable protective immunity from vaccination, sufficient effect of vaccination may sometimes not be obtained. Therefore, development of a means for providing immunity as early and surely as possible has been desired as one of means for preventing occurrence of diseases. Under the circumstances, Sharma et al. reported in ovo inoculation (Patent reference 1) and demonstrated that in ovo inoculation of a Marek's disease (MD) live vaccine could bring about earlier immunization than the conventional vaccination after hatching (Non-patent reference 1). The features of a vaccine for in ovo inoculation include expectation of earlier immunization, reduction in labor as compared to vaccinate after hatching, capacity for mechanization and automatization of inoculation, capacity for more uniform and surer inoculation, and alleviation in stress imposed on chicken.

However, only two have commonly been used for in ovo inoculation, i.e. MD live vaccine and infectious bursal disease (IBD) live vaccine, but most of the other vaccines conventionally used after hatching have not yet been in practical usage for in ovo inoculation. The reason is that most viruses for use in chicken live vaccines, as cultured in chicken embryo primary cells or embryonated chicken eggs, would exert lethal effects on embryo when inoculated in ovo. The situation is the same in case of vaccine strains such as infectious bronchitis (IB) virus or Newcastle disease (ND) virus and, for application of these viruses for in ovo inoculation, some measures must be taken so as to reduce their pathogenicity to embryo.

At present, no ND live vaccine or IB live vaccine are known that are commercially available for suitable in ovo inoculation. For ND live vaccine to be used for in ovo inoculation, the use of a chemical mutagen for producing ND virus mutant Hitchner B1 strain that is non-pathogenic to late embryo (patent reference 2) and the use of ND virus mutant that expresses a low level of V protein (patent reference 3) have been disclosed. However, when these mutants are used as a vaccine, a possibility is not completely deniable that they may have detrimental effects on fowls as well as human beings who ingest the fowls. Therefore, these mutants have not yet become exploited widespread and their evaluation is expected to require quite long term.
Patent reference 1: U.S. Pat. No. 4,458,630
Patent reference 2: U.S. Pat. No. 5,427,791
Patent reference 3: Japanese patent publication No. 2001-069970
Non-patent reference 1: Sharma, J. M., et al., Avian Diseases, 26(1), p. 134-149, 1982

### DISCLOSURE OF THE INVENTION

### (Technical Problem to be Solved by the Invention)

As described above, at present only two have commonly been used for in ovo inoculation, i.e. MD live vaccine and IBD live vaccine. Most of the conventional vaccines used after hatching have not yet been in practical usage for in ovo inoculation since the viruses per se have pathogenicity to embryo and thus pose a safety problem to induce reduction in hatching rate as well as severity in clinical symptoms after hatching by in ovo inoculation. Therefore, some measures must be taken for their application for in ovo inoculation so as to reduce their pathogenicity to embryo e.g. by attenuating the viruses per se. This however is an obstacle difficult to overcome. However, if a vaccine could be developed that is capable of in ovo inoculation for diseases other than MD and IBD as well, such a vaccine is expected to greatly contribute to hygienic measures in fowl industry as enabling acquisition of protective immunity to a wide variety of causative viruses.

### (Means for Solving the Problems)

Under the circumstances, the present inventors have earnestly continued research activities in order to solve the problems described above and as a result have found that, by holding live viruses on a virus-adsorbing agent through adsorption, viral growth in embryonated chicken eggs after in ovo inoculation may be retarded to thereby reduce pathogenicity of the viruses to embryo and that protective immunity may effectively be induced even while live viruses are held on a virus-adsorbing agent through adsorption and thus have completed the present invention.

Thus, the present invention provides a fowl vaccine for in ovo inoculation as described below.
(1) A fowl vaccine for in ovo inoculation comprising a live virus as an antigen characterized by that said vaccine comprises a virus-adsorbing agent.
(2) The vaccine as defined above wherein said virus-adsorbing agent is selected from an aluminum compound, hydroxyapatite, or silica gel.
(3) The vaccine as defined above wherein said aluminum compound is selected from the group consisting of aluminum hydroxide, aluminum phosphate, aluminum chloride, trisodium phosphate aluminum chloride and potassium alum.
(4) The vaccine as defined above wherein said live virus is at least one selected from the group consisting of Marek's disease virus, fowl pox virus, infectious bursal disease virus, Newcastle disease virus, infectious bronchitis virus, infectious laryngotracheitis virus, avian encephalomyelitis virus, chicken anemia virus, avian influenza virus, avian reovirus, avian leukemia virus, reticuloendotheliosis virus, fowl adenovirus, avian pneumovirus and pigeon pox virus.
(5) The vaccine as defined above wherein said fowl is selected from the group consisting of a chicken, a turkey, a guinea fowl, a quail, an ostrich and a pigeon.
(6) A method for immunizing a fowl which comprises administering in ovo the vaccine of any of (1) to (5) above to the fowl in an amount effective for immune production.
(7) The method as defined above wherein said vaccine is administered during the last quarter of incubation of embryo.
(8) A method for preparing a fowl vaccine for in ovo inoculation which comprises mixing a solution of a live vaccine with a virus-adsorbing agent and stirring the resultant mixture to let the live virus held on the virus-adsorbing agent through adsorption.
(9) The method as defined above wherein said virus-adsorbing agent is selected from an aluminum compound, hydroxyapatite, or silica gel.
(10) The method as defined above wherein said aluminum compound is selected from the group consisting of aluminum hydroxide, aluminum phosphate, aluminum chloride, trisodium phosphate aluminum chloride and potassium alum.
(11) The method as defined above wherein said live virus is at least one selected from the group consisting of Marek's disease virus, fowl pox virus, infectious bursal disease virus, Newcastle disease virus, infectious bronchitis virus, infectious laryngotracheitis virus, avian encephalomyelitis virus, chicken anemia virus, avian influenza virus, avian reovirus, avian leukemia virus, reticuloendotheliosis virus, fowl adenovirus, avian pneumovirus and pigeon pox virus.
(12) The method as defined above wherein said fowl is selected from the group consisting of a chicken, a turkey, a guinea fowl, a quail, an ostrich and a pigeon.
(13) A use of a virus-adsorbing agent in a fowl vaccine for in ovo inoculation comprising a live virus as an antigen.
(14) The use as defined above wherein said virus-adsorbing agent is selected from an aluminum compound, hydroxyapatite, or silica gel.
(15) The use as defined above wherein said aluminum phosphate aluminum chloride and potassium alum.
(16) The use as defined above wherein said live virus is at least one selected from the group consisting of Marek's disease virus, fowl pox virus, infectious bursal disease virus, Newcastle disease virus, infectious bronchitis virus, infectious laryngotracheitis virus, avian encephalomyelitis virus, chicken anemia virus, avian influenza virus, avian reovirus, avian leukemia virus, reticuloendotheliosis virus, fowl adenovirus, avian pneumovirus and pigeon pox virus.
(17) The use as defined above wherein said fowl is selected from the group consisting of a chicken, a turkey, a guinea fowl, a quail, an ostrich and a pigeon.
   (More Efficacious Effects than Prior Art) p According to the present invention, by using a virus-adsorbing agent, the use as a vaccine for in ovo inoculation becomes possible in a safe and effective manner for a vaccine strain such as ND virus or IB virus as well as the most viruses having lethal pathogenicity to embryonated chicken eggs, which viruses have been hampered for their use for in ovo inoculation due to a safety problem, i.e. reduction in hatching rate as well as severity in clinical symptoms after hatching by the inoculation.

### BEST MODE FOR CARRYING OUT THE INVENTION

A virus-adsorbing agent as used herein may be any that has a property to hold viruses thereon through adsorption by a physical or chemical action but preferably includes an aluminum compound in a colloidal form, i.e. with dispersion of microparticles in a size ranging from 1 nm to 1 μm in a certain medium. Said aluminum compound includes aluminum hydroxide, aluminum phosphate, aluminum chloride, trisodium phosphate aluminum chloride, potassium alum and the like which may be prepared and used in a conventional manner (Hiroyuki Yuki, Adjuvant and vaccine, Report of Japanese Association of Veterinary Biologics, 22(6), 1-6, 1989).

A live virus as used herein includes a live vaccine virus commonly used for protection of fowl from diseases such as Marek's disease virus, fowl pox virus, infectious bursal disease virus, Newcastle disease virus, infectious bronchitis virus, infectious laryngotracheitis virus, avian encephalomyelitis virus, chicken anemia virus, avian influenza virus, avian reovirus, avian leukemia virus, reticuloendotheliosis virus, fowl adenovirus, avian pneumovirus and pigeon pox virus and may be used each alone or in admixture of two or more thereof.

These live viruses may be prepared by the usual method known in the art (Outline of viral experiment, ed. by alumni association of the National Institute of Health, Maruzen, 1964). Briefly, viruses may be inoculated into a sensitive substrate and, after propagation up to replication in a desired amount of viruses, virus-containing material may be collected. A sensitive substrate may be a variety of substrates capable of viral replication, including embryonated chicken eggs, culture of chicken primary cell embryo such as e.g. chicken embryo fibroblasts (CEF) or chicken kidney cells (CK), or mammal cell line such as VERO cell line, hamster lung (HmLu-1) cell line or baby hamster kidney (BHK) cell line.

A vaccine for in ovo inoculation of the present invention may be prepared by the usual method for preparing a vaccine known in the art using a virus-adsorbing agent (cf. Derek T. O'Hagan et al. ed., Vaccine Adjuvants, Humana Press, 2002). For instance, it may be prepared by mixing viruses with a virus-adsorbing agent previously prepared or by preparing a virus-adsorbing agent in a solution of viruses.

A vaccine for in ovo inoculation of the present invention may be inoculated in ovo by the conventional method (cf. Patent reference 1 supra). In ovo inoculation of a vaccine includes vaccination into embryo contained within an egg. Vaccination may usually be performed at a later stage of embryogenesis, in general, during the last quarter of incubation (embryonated chicken eggs of 15-21 days old) and preferably to embryonated chicken eggs of 18-21 days old.

A vaccine for in ovo inoculation of the present invention may suitably be used in a chicken and also effectively be inoculated to other fowls such as, for instance, a turkey, a guinea fowl, a quail, an ostrich and a pigeon.

A vaccine for in ovo inoculation of the present invention may also be used in admixture with other inactivated vaccines. For mixing, a live vaccine for in ovo inoculation of the present invention and an inactivated vaccine may previously be mixed together or alternatively a live vaccine for in ovo inoculation of the present invention and an inactivated vaccine, each prepared separately, may be mixed when use.

The present invention is explained in more detail by means of the following Examples which are not intended to restrict a scope of the present invention in any sense.

### Example 1

### Preparation of ND Vaccine for in ovo Inoculation Comprising Virus-Adsorbing Agent

A Newcastle disease (ND) vaccine for in ovo inoculation was prepared comprising as a virus-adsorbing agent potassium alum, aluminum hydroxide gel, hydroxyapatite or silica gel. Potassium alum and aluminum hydroxide gel were routinely prepared to give an aluminum content of 2 mg/ml and hydroxyapatite was prepared at 200 mg/ml with a physiological saline, respectively. Each 10 ml of the solution of potassium alum, aluminum hydroxide gel or hydroxyapatite was mixed with 10 ml of a solution of ND virus attenuated D26 strain prepared to give 10^{5.3}EID₅₀/ml with a physiological saline and the mixture was stirred with a stirrer at 4° C. overnight. Also, 1 g of silica gel in powder was mixed with 1 ml of a solution of ND virus attenuated D26 strain prepared with a physiological saline to give 10^{6.3}EID₅₀/ml, thereto was added a physiological saline to make 20 ml in a total amount and the mixture was stirred with a stirrer at 4° C. overnight.

After mixing, in order to verify viral adsorption to each of the virus-adsorbing agents, the vaccines thus prepared were centrifuged at 2500 rpm for 5 minutes and a virus titer remaining in supernatant was measured using embryonated chicken eggs of 11 days old derived from Specific Pathogen Free (hereinafter referred to as “SPF”) chicken. As a result, sufficient viral adsorption could be verified for all of the virus-adsorbing agents. In particular, when potassium alum and hydroxyapatite were used as a virus-adsorbing agent, a virus titer was less than detection limits (Table 1). From the above results, it was assessed that all the virus-adsorbing agents could be used in the present invention.

TABLE 1

| | | | Post-adsorptive |
|---|---|---|---|
| | | | virus titer in |
| | | Pre-adsorptive | supernatant from |
| | Virus-adsorbing | virus titer | centrifuge |
| Group | agent | (EID₅₀/20 ml) | (EID₅₀/20 ml) |
| 1 | potassium alum | 10^{6.3} | <10^{1.8} |
| 2 | Aluminum | 10^{6.3} | 10^{2.2} |
| | hydroxide gel | | |
| 3 | Hydroxyapatite | 10^{6.3} | <10^{1.8} |
| 4 | Silica gel | 10^{6.3} | 10^{2.8} |

### Example 2

### Safety of ND Vaccine for in ovo Inoculation Comprising Virus-Adsorbing Agent in SPF Egg

Using the ND vaccines for in ovo inoculation comprising potassium alum or aluminum hydroxide gel prepared as described in Example 1, each 0.1 ml/egg of the vaccine was inoculated in ovo to SPF embryonated chicken eggs of 18 days old (Groups 1 and 2), as reported by Sharma et al. (cf. Non-patent reference 1 supra). As a control, a test group was set that comprised the virus alone but without a virus-adsorbing agent (Group 3). As a result, as shown in Table 2, the group of the ND vaccines for in ovo inoculation comprising a virus-adsorbing agent exhibited a hatching rate significant difference from that of the test group without a virus-adsorbing agent (test for statistical significance to the test group without a virus-adsorbing agent by Fisher's exact probability test) Besides, the group of the ND vaccines for in ovo inoculation comprising a virus-adsorbing agent also exhibited a survival rate at 2 Weeks after hatching significant difference from that of the test group without a virus-adsorbing agent (Table 3).

TABLE 2

| | Virus titer | Virus- | No. of | No. of | |
|---|---|---|---|---|---|
| | in dose | adsorbing | eggs | chickens | Hatching |
| Group | (EID₅₀/0.1 ml) | agent | tested | hatched | rate (%) |
| 1 | 10^{2.0} | Potassium | 50 | 37 | 74**** |
| | | alum | | | |
| 2 | | Aluminum | 50 | 28 | 56* |
| | | hydroxide | | | |
| | | gel | | | |
| 3 | | — | 50 | 16 | 32 |

| | | | | |
|---|---|---|---|---|
| 4 | No inoculation | 100 | 88 | 88 |

(Note)
*P < 0.05,
****P < 0.0001

TABLE 3

| | | | | No. of survived | |
|---|---|---|---|---|---|
| | Virus titer | Virus- | No. of | chickens at 2 | Survival rate |
| | in dose | adsorbing | chickens | Weeks after | (%) at 2 Weeks |
| Group | (EID₅₀/0.1 ml) | agent | tested | hatching | after hatching |
| 1 | 10^{2.0} | Potassium | 37 | 33 | 89** |
| | | alum | | | |
| 2 | | Aluminum | 28 | 22 | 79* |
| | | hydroxide | | | |
| | | gel | | | |
| 3 | | — | 16 | 7 | 44 |

| | | | | |
|---|---|---|---|---|
| 4 | No inoculation | 80 | 80 | 100 |

(Note)
*P < 0.05,
**P < 0.01

### Example 3

### Efficacy of ND Vaccine for in ovo Inoculation Comprising Virus-Adsorbing Agent in SPF Egg

For evaluating efficacy of the test groups subject to Example 2, serum was taken at 3 Weeks after hatching and haemagglutination inhibition test (HI test) was performed with hemagglutinin of ND virus (Juridical Foundation The Chemo-Sero-Therapeutic Research Institute) in accordance with the instructions attached thereto. It is known in general that challenge by velogenic ND virus Sato strain may be protected with HI antibody titer of 5-fold or more. Therefore, the HI antibody titer in the group of the ND vaccines for in ovo inoculation comprising a virus-adsorbing agent well surpassed the protection level (Table 4).

TABLE 4

| | | | | HI antibody |
|---|---|---|---|---|
| | | | | titer (GM |
| | Virus titer | Virus- | No. of | value, |
| | in dose | adsorbing | chickens | fold), 3 |
| Group | (EID₅₀/0.1 ml) | agent | tested | weeks old |
| 1 | 10^{2.0} | Potassium | 20 | 52.8 [100]¹⁾ |
| | | alum | | |
| 2 | | Aluminum | 20 | 52.8 [100] |
| | | hydroxide gel | | |
| 3 | | — | 6 | 71.3 [100] |

| | | | |
|---|---|---|---|
| 4 | No inoculation | 10 | 2.5 [0] |

(Note)
¹⁾In the parenthesis, antibody positive rate (%) is shown. Five-fold or more is regarded as positive.

### Example 4

### Preparation of IB and FP Vaccines for in ovo Inoculation Comprising Virus-Adsorbing Agent

Infectious bronchitis (IB) and fowl pox (FP) vaccines for in ovo inoculation were prepared comprising potassium alum as a virus-adsorbing agent. Potassium alum was routinely prepared to give an aluminum content of 2 mg/ml with a physiological saline. 10 ml of the solution of potassium alum was mixed with 10 ml of a solution of IB virus TM-86w strain prepared to give 10^{5.8}EID₅₀/ml with a physiological saline or with 10 ml of a solution of pigeon pox (PP) virus KIII strain derived from Nakano strain prepared to give 10^{4.3}TCID₅₀/ml with a physiological saline and the mixture was stirred with a stirrer at 4° C. overnight After mixing, in order to verify viral adsorption to the virus-adsorbing agent, the vaccines thus prepared were centrifuged at 2500 rpm for 5 minutes and a virus titer remaining in supernatant was measured using embryonated chicken eggs of 11 days old derived from SPF chicken. As a result, sufficient viral adsorption could be verified for both IB and PP (Table 5).

TABLE 5

| | | | Post-adsorptive virus |
|---|---|---|---|
| | | Pre-adsorptive | titer in supernatant |
| | | virus titer | from centrifuge |
| Group | Virus | (E[TC]ID₅₀/20 ml) | (E[TC]ID₅₀/20 ml) |
| 1 | IB | 10^{6.8} | 10^{3.2} |
| 2 | PP | 10^{5.3} | 10^{3.4} |

### Example 5

### Safety of IB Vaccine for in ovo Inoculation Comprising Virus-Adsorbing Agent in SPF Egg

Using the IB vaccine for in ovo inoculation comprising potassium alum prepared as described in Example 4, each 0.1 ml/egg of the vaccine was inoculated in ovo to SPF embryonated chicken eggs of 18 days old (Group 1), as reported by Sharma et al. As a control, a test group was set that comprised the virus alone but without a virus-adsorbing agent (Group 2). As a result, as shown in Table 6, the group of the IB vaccine for in ovo inoculation comprising a virus-adsorbing agent exhibited a hatching rate significant difference from that of the test group without a virus-adsorbing agent (test for statistical significance to the test group without a virus-adsorbing agent by Fisher's exact probability test).

TABLE 6

| | Virus titer | Virus- | No. of | No. of | |
|---|---|---|---|---|---|
| | in dose | adsorbing | eggs | chickens | Hatching |
| Group | (EID₅₀/0.1 ml) | agent | tested | hatched | rate (%) |
| 1 | 10^{2.5} | Potassium | 50 | 32 | 64**** |
| | | alum | | | |
| 2 | | — | 50 | 11 | 22 |

| | | | | |
|---|---|---|---|---|
| 3 | No inoculation | 100 | 88 | 88 |

(Note)
****P < 0.0001

### Example 6

### Efficacy of IB Vaccine for in ovo Inoculation Comprising Virus-Adsorbing Agent in SPF Egg

For evaluating efficacy of the test groups subject to Example 5, serum was taken at 4 Weeks after hatching and a titer of a neutralizing antibody was measured. Also, at 4 Weeks after hatching, IB virus TM-86EC strain was inoculated intratracheally at 10^{3.5}EID₅₀/0.1 ml per chicken and at 4 Days after inoculation the trachea was removed to observe ciliary activity in the trachea. As a result, as shown in Table 7, the sufficient effect could be verified with 2.0-fold or more of the titer of a neutralizing antibody and 0% of a stoppage rate of the ciliary activity in the trachea.

TABLE 7

| | | | | Antibody titer | Stoppage rate of |
|---|---|---|---|---|---|
| | Virus titer | Virus- | No. of | (GM value, | ciliary activity |
| | in dose | adsorbing | chickens | exponent) | in trachea (%) |
| Group | (EID₅₀/0.1 ml) | agent | tested | 4 Weeks old | 4 Weeks old |
| 1 | 10^{2.5} | Potassium | 17-18 | 3.09 [100]¹⁾ | 0 |
| | | alum | | | |
| 2 | | — | 7-8 | 2.64 [71] | 0 |

| | | | | |
|---|---|---|---|---|
| 3 | No inoculation | 10-20 | 0 [0] | 90 |

(Note)
¹⁾In the parenthesis, antibody positive rate (%) is shown. Antibody titer of 2.0 or more is regarded as positive.

### Example 7

### Safety of FP Vaccine for in ovo Inoculation Comprising Virus-Adsorbing Agent in SPF Egg

Using the FP vaccine for in ovo inoculation comprising potassium alum prepared as described in Example 4, each 0.1 ml/egg of the vaccine was inoculated in ovo to SPF embryonated chicken eggs of 18 days old (Group 1), as reported by Sharma et al. As a control, a test group was set that comprised the virus alone but without a virus-adsorbing agent (Group 2). As a result, as shown in Table 8, the group of the FP vaccine for in ovo inoculation comprising a virus-adsorbing agent exhibited a hatching rate significant difference from that of the test group without a virus-adsorbing agent (test for statistical significance to the test group without a virus-adsorbing agent by Fisher's exact probability test).

TABLE 8

| | Virus titer | Virus- | No. of | No. of | |
|---|---|---|---|---|---|
| | in dose | adsorbing | eggs | chickens | Hatching |
| Group | (EID₅₀/0.1 ml) | agent | tested | hatched | rate (%) |
| 1 | 10^{3.0} | Potassium | 50 | 37 | 74** |
| | | alum | | | |
| 2 | | — | 50 | 22 | 44 |

| | | | | |
|---|---|---|---|---|
| 3 | No inoculation | 100 | 88 | 88 |

(Note)
**P < 0.01

### Example 8

### Efficacy of FP Vaccine for in ovo Inoculation Comprising Virus-Adsorbing Agent in SPF Egg

For evaluating efficacy of the test groups subject to Example 7, serum was taken at 3 Weeks after hatching and a titer of a fluorescence antibody (FA) was measured. Also, at 3 Weeks after hatching, FP virus Nishigahara strain at 10^{4.0}TCID₅₀/0.1 ml per chicken was dropped to the femoral region after removal of feather and spread by scrubbing with a toothbrush. At 3 Weeks after inoculation, clinical symptoms were observed and the presence of pox formation, scab and ulcer was checked. As a result, the group of the FP vaccine for in ovo inoculation comprising a virus-adsorbing agent exhibited an FA positive rate and a rate of protection against challenge by FP virus virulent strain both better than those of the test group without a virus-adsorbing agent (Table 9).

TABLE 9

| | Virus titer | Virus- | No. of | FA positive | Rate of |
|---|---|---|---|---|---|
| | in dose | adsorbing | chickens | rate¹⁾ (%) | protection (%) |
| Group | (TCID₅₀/0.1 ml) | agent | tested | 3 Weeks old | 3 Weeks old |
| 1 | 10^{3.0} | Potassium | 17-20 | 47 | 55 |
| | | alum | | | |
| 2 | | — | 11-13 | 9 | 23 |

| | | | | |
|---|---|---|---|---|
| 3 | No inoculation | 10-20 | 0 | 0 |

(Note)
¹⁾FA titer of 20-fold or more is regarded as positive.

### INDUSTRIAL APPLICABILITY

The fowl vaccine for in ovo inoculation according to the present invention, by holding live viruses on a virus-adsorbing agent through adsorption, may retard viral growth in embryonated chicken eggs after in ovo inoculation to thereby reduce pathogenicity of the viruses to embryo and thus is highly safe not to induce reduction in a hatching rate as well as severity in clinical symptoms after hatching by in ovo inoculation. Furthermore, the fowl vaccine for in ovo inoculation according to the present invention, comprising live viruses as an antigen, may exert protective effects against diseases caused by said viruses like a conventional live vaccine. Accordingly, with the fowl vaccine for in ovo inoculation according to the present invention, a vaccine with any kind of natural live viruses may be prepared not limited to the currently prevailing specific live vaccines such as MD vaccine and IBD vaccine to thereby enable acquisition of a broad range of protective immunity to lethal causative viruses of a wide variety of diseases. It is thus expected that the fowl vaccine for in ovo inoculation according to the present invention would much contribute to the hygienic measure in fowl industry.

1. A fowl vaccine for in ovo inoculation comprising a live virus as an antigen characterized by that said vaccine comprises a virus-adsorbing agent.

2. The vaccine of claim 1 wherein said virus-adsorbing agent is selected from an aluminum compound, hydroxyapatite, or silica gel.

3. The vaccine of claim 2 wherein said aluminum compound is selected from the group consisting of aluminum hydroxide, aluminum phosphate, aluminum chloride, trisodium phosphate aluminum chloride and potassium alum.

4. The vaccine of claim 1, wherein said live virus is at least one selected from the group consisting of Marek's disease virus, fowl pox virus, infectious bursal disease virus, Newcastle disease virus, infectious bronchitis virus, infectious laryngotracheitis virus, avian encephalomyelitis virus, chicken anemia virus, avian influenza virus, avian reovirus, avian leukemia virus, reticuloendotheliosis virus, fowl adenovirus, avian pneumovirus and pigeon pox virus.

5. The vaccine of claim 1, wherein said fowl is selected from the group consisting of a chicken, a turkey, a guinea fowl, a quail, an ostrich and a pigeon.

6. A method for immunizing a fowl which comprises administering in ovo the vaccine of claim 1 to the fowl in an amount effective for immune production.

7. The method of claim 6 wherein said vaccine is administered during the last quarter of incubation of embryo.

8. A method for preparing a fowl vaccine for in ovo inoculation which comprises mixing a solution of a live vaccine with a virus-adsorbing agent and stirring the resultant mixture to let the live virus held on the virus-adsorbing agent through adsorption.

9. The method of claim 8 wherein said virus-adsorbing agent is selected from an aluminum compound, hydroxyapatite, or silica gel.

10. The method of claim 9 wherein said aluminum compound is selected from the group consisting of aluminum hydroxide, aluminum phosphate, aluminum chloride, trisodium phosphate aluminum chloride and potassium alum.

11. The method of claim 8, wherein said live virus is at least one selected from the group consisting of Marek's disease virus, fowl pox virus, infectious bursal disease virus, Newcastle disease virus, infectious bronchitis virus, infectious laryngotracheitis virus, avian encephalomyelitis virus, chicken anemia virus, avian influenza virus, avian reovirus, avian leukemia virus, reticuloendotheliosis virus, fowl adenovirus, avian pneumovirus and pigeon pox virus.

12. The method of claim 8, wherein said fowl is selected from the group consisting of a chicken, a turkey, a guinea fowl, a quail, an ostrich and a pigeon.

13-17. (canceled)
